(19) United States
(12) Patent Application Publication
(10) Pub. No.: US 2023/0381454 A1
(43) Pub. Date: Nov. 30, 2023

(54) AORTO OSTIAL FLUID DIRECTING DEVICE

(71) Applicants: BOSTON SCIENTIFIC SCIMED, INC., MAPLE GROVE, MN (US); REGENTS OF THE UNIVERSITY OF MINNESOTA, MINNEAPOLIS, MN (US)

(72) Inventors: TINA MARIE LENNEMAN, OTSEGO, MN (US); DENNIS A. BOISMIER, SHOREWOOD, MN (US); KYLE HARISH SRIVASTAVA, Wayzata, MN (US); KEVIN J. GOODWIN, MINNEAPOLIS, MN (US); FELIX LANDAETA, MINNEAPOLIS, MN (US); PAIGE V. TRACY, SOUTH ST. PAUL, MN (US)

(73) Assignees: BOSTON SCIENTIFIC SCIMED, INC., MAPLE GROVE, MN (US); REGENTS OF THE UNIVERSITY OF MINNESOTA, MINNEAPOLIS, MN (US)

(21) Appl. No.: 18/232,100

(22) Filed: Aug. 9, 2023

Related U.S. Application Data
(63) Continuation of application No. 16/562,220, filed on Sep. 5, 2019.
(60) Provisional application No. 62/727,300, filed on Sep. 5, 2018.

(51) Int. Cl. A61M 25/00; A61M 25/01; A61F 2/844; A61B 6/00; A61F 2/82; A61F 2/95; A61B 17/00; A61M 25/02

(52) U.S. Cl. CPC A61M 25/0082; A61M 25/0105; A61M 25/003; A61M 25/0074; A61F 2/844; A61B 6/504; A61F 2002/821; A61F 2/95; A61M 2025/0096; A61M 2025/0166; A61B 2017/00243; A61M 25/02; A61M 2025/0073

(57) ABSTRACT

A fluid directing device may include a catheter and a skirt. The catheter may define a lumen and may have a distal end and a proximal end. The skirt may be attached to a distal region of the catheter and may encircle the catheter. The skirt may have a proximal end attached to the catheter, and a free distal end, the skirt having a sidewall extending between the proximal and distal ends thereof, the skirt configured to move between a collapsed state and an expanded state in which the sidewall extends radially away from the catheter, the sidewall defining an interior chamber in the expanded state, wherein the skirt is configured to prevent contrast media that exits the catheter lumen from passing through the sidewall.

### CROSS-REFERENCE TO RELATED APPLICATIONS

This application is a continuation of U.S. patent application Ser. No. 16/562,220, filed Sep. 5, 2019, which claims the benefit of priority under 35 U.S.C. § 119 to U.S. Provisional Application Ser. No. 62/727,300, filed Sep. 5, 2018, the entirety of which is incorporated herein by reference.

### TECHNICAL FIELD

The present disclosure pertains to medical devices, and methods for manufacturing medical devices. More particularly, the present disclosure pertains to medical devices for directing fluid.

### BACKGROUND

Balloon angioplasty and stenting procedures (percutaneous transluminal coronary angioplasty, PTCA) of atherosclerotic lesions in the ostia of arteries branching off from aorta have proven to be difficult. In addition, percutaneous transluminal angioplasty in the ostia of arteries branching off from aorta has been associated with increased risk of operative problems such as ostial trauma, inability to inflate balloon with appropriate catheter support, and an increased need for intracoronary manipulation. Many of the difficulties and risks encountered with conventional techniques used in these procedures can be traced to difficulties in visualizing the geometrical shape of the ostia of arteries. Standard visualization techniques such as X-Ray imaging may be used. The use of radiopaque contrast media may provide increased visualization of the anatomical structures and lesions, however the use of contrast media may involve complications regarding directing the contrast media to the desired location without overflow to other regions of the body. This impediment of visualization may lead to inaccuracies in balloon angioplasty, deployment of stents, and other complications. For example, if a stent is not positioned correctly and extends beyond the ostium of a vessel, cannulation of another guide wire into the vessel and subsequent access to the vessel becomes extremely difficult. Additionally, if a stent does not appropriately cover the atherosclerotic lesion, the risk of restenosis increases considerably. Thus, accurate placement of a stent at the ostium of an aortic arterial branch is essential. Additionally, there is a need for devices and procedures to reduce the amount of contrast media introduced into the body. Contrast-induced nephropathy (CIN) is a serious complication of angiographic procedures resulting from the administration of contrast media, and may result in renal injury. Recently, devices or methods that assist in placement of stents at ostium of blood vessels have been developed. However, many of these techniques may not provide sufficient information for accurate lesion identification and stent placement, may not be easy to use, and may require significant contrast media usage. Thus, there exists a need in the art to develop medical devices that visualize endoluminal ostial geometry of blood vessels and assist in anchoring a catheter to the aortic wall with a reduced amount of contrast media.

### BRIEF SUMMARY

This disclosure provides design, material, manufacturing method, and use alternatives for medical devices. An example fluid directing device comprises a catheter defining a lumen and having a distal end and a proximal end, and a skirt attached to and encircling a distal region of the catheter, the skirt having a proximal end attached to the catheter, and a free distal end, the skirt having a sidewall extending between the proximal and distal ends thereof, the skirt configured to move between a collapsed state and an expanded state in which the sidewall extends radially away from the catheter, the sidewall defining an interior chamber in the expanded state, wherein the skirt is configured to prevent contrast media that exits the catheter lumen from passing through the sidewall.

Alternatively or additionally to the embodiment above, the fluid directing device further comprises an attachment member disposed on the distal end of the skirt.

Alternatively or additionally to the embodiment above, the attachment member includes a plurality of hooks configured to engage an ostium of a vessel.

Alternatively or additionally to the embodiment above, the skirt includes an inner side wall and an outer sidewall with a space therebetween, wherein the attachment member includes a vacuum lumen within the catheter in fluid communication with the space between the inner and outer sidewalls.

Alternatively or additionally to the embodiment above, the sidewall is permeable to blood and impermeable to contrast media.

Alternatively or additionally to the embodiment above, the sidewall is permeable to fluid flow in a single direction, permitting fluid to flow through the sidewall from outside the skirt into the interior chamber, but preventing fluid flowing through the sidewall from the interior chamber to outside the skirt.

Alternatively or additionally to the embodiment above, the sidewall is impermeable to blood and contrast media.

Alternatively or additionally to the embodiment above, the sidewall includes electrically actuatable pores allowing the sidewall to be permeable or impermeable, depending on an activation state of the pores.

Alternatively or additionally to the embodiment above, the proximal end of the skirt is attached to the distal end of the catheter.

Alternatively or additionally to the embodiment above, the proximal end of the skirt is attached proximal of the distal end of the catheter, such that the distal end of the catheter extends within the interior chamber of the skirt or distal of the skirt.

Alternatively or additionally to the embodiment above, the skirt further comprises a support structure configured to bias the skirt in the expanded state.

Alternatively or additionally to the embodiment above, the support structure includes a plurality of struts extending between the proximal end and distal end of the skirt.

Alternatively or additionally to the embodiment above, the fluid directing device further comprises at least one sensor configured to determine proximity to tissue.

Alternatively or additionally to the embodiment above, the distal end of the skirt is made of a soft elastomeric material.

Another example fluid directing device comprises a catheter defining a lumen, and a skirt attached to and encircling a distal end of the catheter, the skirt having a free distal end extending distal of the distal end of the catheter, the skirt configured to move between a collapsed state and an expanded state in which the skirt extends radially away from the catheter, the skirt defining an interior chamber in the expanded state, wherein the skirt is configured to prevent contrast media that exits the catheter lumen from passing through the skirt.

Alternatively or additionally to the embodiment above, the fluid directing device further comprises an attachment member disposed on the distal end of the skirt.

Alternatively or additionally to the embodiment above, the attachment member includes a plurality of hooks configured to engage an ostium of a vessel.

Alternatively or additionally to the embodiment above, the skirt includes an inner sidewall and an outer sidewall with a space therebetween, wherein the attachment member includes a vacuum lumen within the catheter in fluid communication with the space between the inner and outer sidewalls.

Alternatively or additionally to the embodiment above, the skirt is permeable to blood and impermeable to contrast media.

An example method of imaging a vessel ostium comprises advancing a fluid directing device intravascularly to the vessel ostium, wherein the fluid directing device includes a catheter with a skirt attached at a distal end thereof, the skirt defined by a sidewall configured to move between a collapsed state and an expanded state in which the sidewall extends radially away from the catheter, expanding the skirt to the expanded state, attaching a distal end of the skirt over the vessel ostium, and delivering contrast media through the catheter and skirt, wherein the skirt prevents contrast media from passing through the sidewall.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a perspective view of an example fluid directing device;

FIG. 2 is a perspective view of an example fluid directing device;

FIG. 3 is a perspective view of an example fluid directing device in place over an ostium;

FIG. 4 is a cross sectional view of an example fluid directing device in place over an ostium;

FIG. 5 is a cross sectional view of an example fluid directing device in place over an ostium; and

FIG. 6 is a partial cross sectional view of an example fluid directing device in place over an ostium.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g. 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, is or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

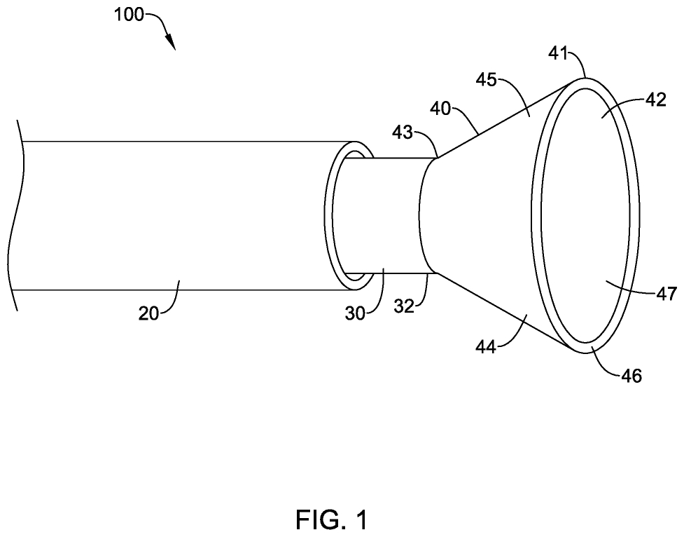

FIG. 1 illustrates a fluid directing device 100 including a delivery shaft 20 and a catheter 30 with a skirt 40 disposed at a distal end of the catheter 30. The catheter 30 may function as a device catheter, having at least one lumen sized to allow passage therethrough of conventional lesion treating, sensing, and/or visualizing instruments. The catheter 30 also allows for delivery of contrast media. In some embodiments, the catheter 30 may have separate lumens for instruments and contrast media. The skirt 40 may be formed of a sidewall 45 having a free distal end 41 and a proximal end 43 encircling and attached to the distal region of the catheter 30. In some embodiments, the skirt 40 may be attached to the distal end 32 of the catheter 30, as shown in FIG. 1. The skirt 40 may be flexible, moving between a collapsed state during delivery, in which the skirt 40 is folded or wrapped to fit inside the delivery shaft 20, to an expanded or deployed state in which the sidewall 45 extends radially away from the catheter 30, as shown in FIG. 1. The sidewall may define an interior chamber 47 in the expanded state. The distal end 41 of the skirt may have a circumference sized to be larger than the circumference of the ostium with which the device will be used. In the deployed configuration, the skirt 40 may have a conical shape, as shown in FIG. 1. The tapered sidewall 45 may act as a ramp to direct contrast medium into the ostium of a vessel to be imaged. In other embodiments, the skirt may have a cylindrical or semi-spherical shape.

In some embodiments, the distal end 41 of the skirt 40 may include an attachment member configured to anchor the catheter 30 to the aortic wall and form a seal around the ostium. The skirt sidewall 45 may include an inner sidewall 42, an outer sidewall 44, and a space 46 therebetween, as shown in FIG. 1. The attachment member may include a vacuum lumen within the catheter 30 in fluid communication with a vacuum source and the space 46 between the inner sidewall 42 and the outer sidewall 44. When the distal end 41 of the skirt 40 is placed against the vessel wall surrounding an ostium, the vacuum source may be activated to hold the fluid directing device 100 in place.

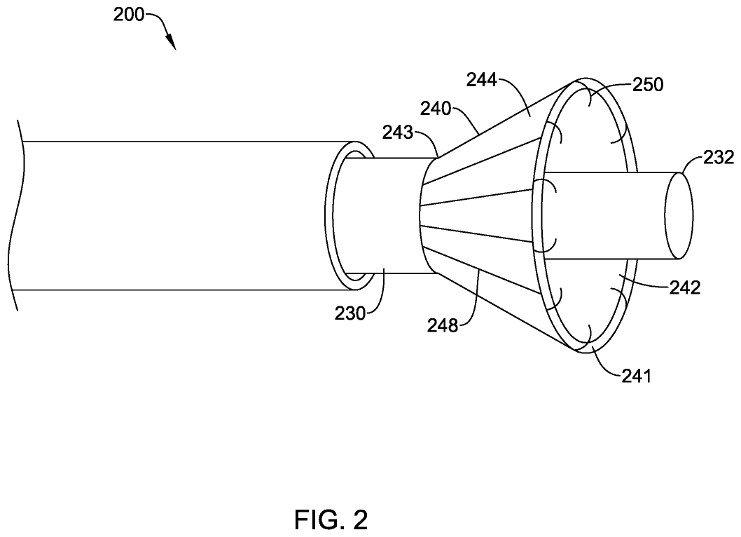

In other embodiments, the fluid directing device 200 may include a catheter 230 with a skirt 240 having a plurality of hooks 250 disposed on the distal end 241 of the skirt 240 as the attachment member, as shown in FIG. 2. The skirt 240 may be attached proximal of the distal end 232 of the catheter 230. When the skirt 240 is placed over an ostium, the distal end 232 of the catheter 230 may extend into the vessel, directing instruments and/or contrast media directly into the vessel. The distal end 232 of the catheter 230 may also provide a mechanical self-centering feature for aligning the skirt 240 with the ostium. It will be understood that the location of the skirt 40, 240 relative to the distal end 32, 232 of the catheter 30, 230 and the attachment members illustrated in FIGS. 1 and 2 may be interchanged.

In some embodiments the skirt 40, 240 may include a support structure to facilitate expansion. As shown in FIG. 2, the support structure may include a plurality of struts 248 extending from the proximal end 243 of the skirt 240 to the distal end 241 of the skirt 240. In some embodiments, the struts 248 may be formed of spring steel, a rigid plastic, or a shape memory metal, such as nitinol. The struts 248 may be biased in the expanded state. The struts 248 may be disposed on the outer sidewall 244 of the skirt 240, on inner sidewall 242 of the skirt 240, or the struts 248 may be disposed within the sidewall defining the skirt 240. In other embodiments, the support structure may include circular supports, or a spiral support (not shown). It will be understood that the skirt 40 illustrated in FIG. 1 may also include the support structure.

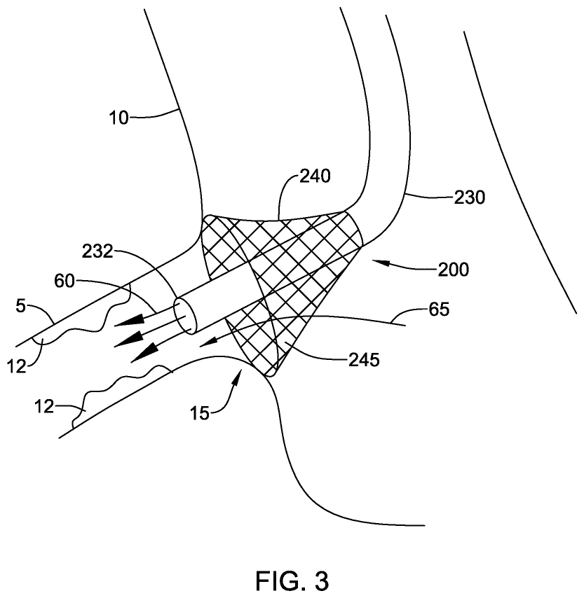

FIG. 3 illustrates fluid directing device 200 in place against the ostium 15 leading from the aorta 10 into the right coronary artery 5. The fluid directing device 200 may improve visualization and treatment of ostial lesions 12 by directing contrast media to cardiac circulation. Treatment may be affected not only within and around the right coronary artery, but also the left coronary artery, left anterior descending artery, left circumflex artery, or any other vessel accessible by the assembly. In some embodiments the fluid directing device 200 may be delivered transcutaneously via the femoral artery or radial artery and deployed at the coronary ostium 15.

Embodiments having the distal end 232 of the catheter 230 extending distally beyond the skirt 240 may allow contrast media, indicated by arrows 60, to be delivered through the catheter 230 directly into the coronary artery 5. This structure may aid in controlling the flow of contrast media, helping prevent contrast media from flowing into the aorta. In some embodiments, the skirt 240 may be configured to allow blood flow, indicated by arrow 65, from the aorta, through the sidewall 245 of the skirt 240, and into the coronary artery 5.

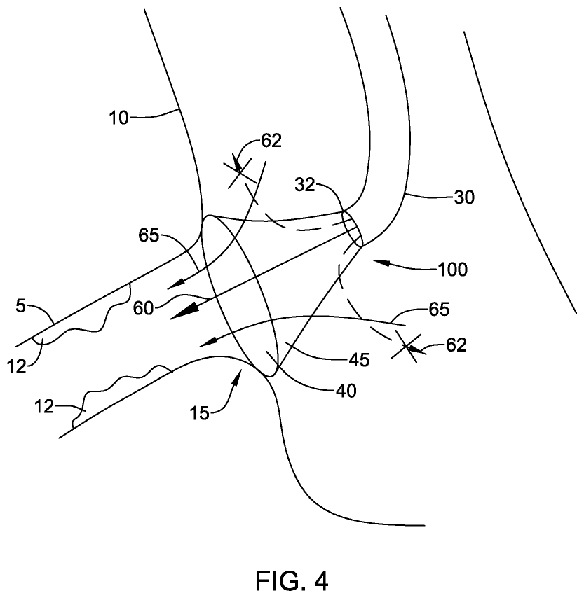

FIG. 4 illustrates fluid directing device 100 in place over the ostium 15. In this embodiment, the skirt 40 is attached to the distal end 32 of the catheter 30. Contrast media may be delivered through the catheter 30 and into the coronary artery 5. If the skirt 40 were permeable to the contrast media, some of the contrast media could flow through the sidewall 45 of the skirt and into the aorta, shown by arrows 62. This may require the use of an increased amount of contrast media to obtain the desired visualization of lesions 12 in the coronary artery 5. This increase in amount of contrast media may cause complications arising from the necessity of the body to clear the contrast media. In some embodiments, the skirt 40 may be configured to allow blood flow 65 through the sidewall 45 of the skirt 40, while preventing the flow of contrast media, indicated by arrows 62, from flowing through the sidewall 45. This may be achieved with a skirt 40 that allows one way fluid flow through the sidewall 45, from outside the skirt 40 to inside the skirt 40. In this manner, blood may flow from the aorta 10 through the skirt sidewall 45 and into the coronary artery 5. Contrast media flowing from the catheter 30 is prevented from flowing is through the sidewall 45 an into the aorta 10, directing the contrast media into the coronary artery 5 where it is needed to aid in imaging lesions 12. In other embodiments, the selective direction of contrast media into the coronary artery 5 illustrated in FIG. 4 is achieved with a skirt 40 that is permeable to blood, allowing blood to flow in either direction through the sidewall 45, but is impermeable to contrast media. As the contrast media flows from the catheter 30, it cannot flow through the sidewall 45 and is thus directed into the coronary artery 5. The skirt 40 thus contains the contrast media to the coronary artery 5. This may have the advantage of reducing the amount of contrast media required for the imaging procedure. The selective permeability of the skirt 40 may be achieved by selecting the pore size of the material forming the skirt to allow blood to pass through but block contrast media.

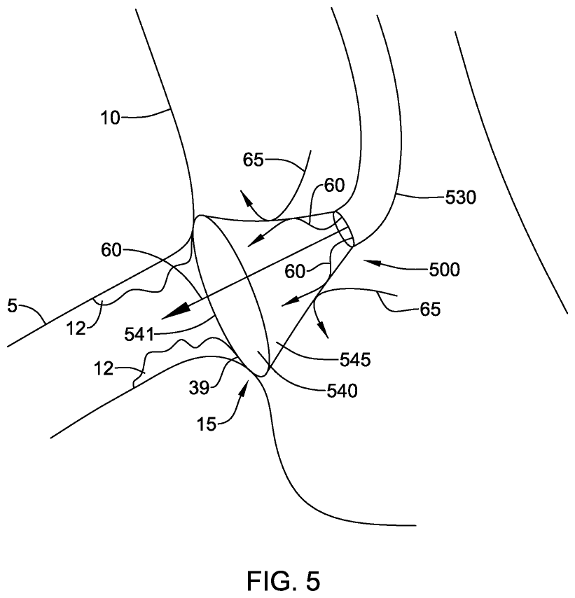

In other embodiments, the skirt 540 may be impermeable to both blood and contrast media. As shown in FIG. 5, when the fluid directing device 500 is in place over the ostium blood flow 65 from the aorta 10 may be prevented from passing through the skirt sidewall 545 and entering the coronary artery 5. Contrast media 60 leaving the catheter 530 is also prevented from passing through the skirt sidewall 545, and instead is directed into the coronary artery 5. In this embodiment, oxygenated fluid may be delivered through the catheter 530 to flow into the coronary artery 5 during the imaging procedure. The oxygenated fluid may be clear and may displace blood from the inner chamber of the skirt and ostium 15, allowing for improved visualization of lesions 12. Oxygenated fluid may be directed through the catheter 530 at the same time as contrast media, or the fluids may be alternated. Continuous flow of oxygenated fluid may be maintained, or it may be pumped temporarily or intermittently until a clear view of the lesions 12 is achieved. When blood flow is desired, the skirt 540 may be withdrawn from the ostium 15.

Additional imaging elements may be used in combination with the fluid directing device 100. For example, imaging elements for forward looking ultrasound, intravascular ultrasound, optical coherence tomography, a video camera, etc., may be passed through the catheter to aid in visualizing the lesions 12. In some embodiments, a radiopaque mesh (not shown) may be disposed over the distal end 32 of the skirt 40 to aid in visualizing the ostial opening.

In some embodiments, the permeability of the skirt 40 may be effectively turned on and off. For example, the skirt sidewall 45 may be formed of a material including electrically actuatable pores. One or more leads (not shown) may extend from the skirt 40 through the catheter 30. The electrically actuatable pores may be biased closed, and when the skirt is expanded and in position over the ostium 15, a current may be transmitted through the leads to open the pores and allow fluid to flow through the skirt sidewall 45.

The skirt 40 may be made of a variety of materials. In the following discussion the skirt 40 will be referenced, however it will be understood that the materials discussed will apply to any embodiment of skirt 40, 240, 540. In some embodiments, the skirt 40 may be made of a flexible biocompatible material including but not limited to, e.g., polymer, plastic, fabric, or metal. The material used for the skirt 40 may depend on the type of imaging to be used. X-ray, computed tomography (CT), magnetic resonance (MR) and ultrasound imaging procedures may dictate a particular material to allow the skirt 40 to be visible or invisible in the image, as desired. The skirt 40 may be formed from a woven or knitted fabric, or may be a continuous membrane which may or may not have pores. In some embodiments, the distal end 41 of the skirt 40 may be made of a soft elastomeric material such as a soft silicone or polyurethane, to help the distal end 41 conform to an uneven or rough underlying anatomical tissue surface, such as forming a seal around the ostium.

In some embodiments, the distal region of the catheter 30, 230, 530 and/or the skirt 240, 540 may include one or more sensor 39 to aid in detecting the position of the skirt 40, 240, 540 relative to the ostium 15. For example, one or more sensor 39 may be disposed on the distal end 541 of the skirt 540, as shown in FIG. 5, and the sensor 39 may be configured and arranged to distinguish between blood contact and tissue contact. In addition, a sensor may be configured to detect proximity to tissue. In at least some examples, a sensor may be configured as an electrode to measure impedance which may be used to detect proximity and/or contact with the vessel walls. In some examples, bipolar electrodes including a positive electrode and a negative electrode may be used, which may eliminate the need for a ground pad or electrode. In other embodiments an imaging sensor may be provided to identify the center of the ostium 15.

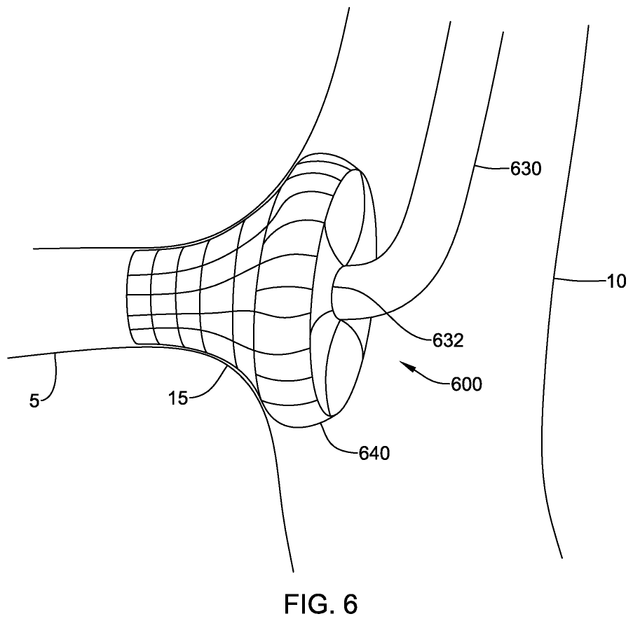

FIG. 6 illustrates another embodiment of fluid directing device 600, in which the skirt is a mushroom-shaped stent 640 that may function in the same manner as the skirts 240, 540 to direct contrast media into the coronary artery 5 while preventing backflow of contrast media into the aorta 10. The mushroom shape of the stent 640 may be deployed partially in the coronary artery 5 and partially in the aorta 10, thereby filling a funnel-shaped ostium 15. The stent 640 may be self-expanding and made of a shape memory material such as nitinol, or it may be expandable with a balloon disposed through the catheter 630. The stent 640 may be attached to the distal end 632 of the catheter 630, as shown in FIG. 6. In other embodiments, the stent 640 may be attached proximal of the distal end of the catheter 630, similar to the skirt 240 shown in FIG. 2. The stent 640 may be formed from an expandable mesh that allows blood to flow through but prevents contrast media from flowing through, as described above with regard to the skirt 40 shown in FIG. 4. In other embodiments, the stent 640 may be impermeable to both blood and contrast media as described above with regard to the skirt 540 shown in FIG. 5. In this embodiment, oxygenated fluid may be delivered through the catheter 630 and into the coronary artery 5. In other embodiments, the stent 640 may include a cover providing the above permeability characteristics. The cover may be formed of a similar material as the skirts 40, 250, 540 described above.

Once the fluid directing device 100, 200, 500, 600 is in place over the ostium 15, as shown in FIGS. 3-6, and contrast media has been delivered to determine the location of lesions 12, a treatment device may be delivered through the catheter 30, 230, 530, 630. In some examples, a stent delivery assembly may be delivered and the stent expanded at the location of the lesion. The stent delivery assembly may include a stent disposed over an inflatable balloon. Additional contrast medium may be delivered through the catheter 30, 230, 530, 630 during deployment of the stent to assure desired placement. The stent may be deployed only in the coronary artery 5 or it may be deployed partially in the coronary artery 5 and partially in the aorta 10. Once the stent or other treatment device is deployed or utilized in treating the lesion, these devices may be withdrawn through the catheter 30, 230, 530, 630 and then the catheter 30, 230, 530, 630 may be withdrawn from the ostium.

Some suitable but non-limiting materials that can be used for the various components of the fluid directing device 100, 200, 500, 600 including the catheter 30, 230, 530, 630 and the skirt 40, 240, 540, 640 (and/or other systems disclosed herein) and the various elements thereof disclosed herein may include those commonly associated with medical devices.

In some embodiments, the catheter 30, 230, 530, 630 and the skirt 40, 240, 540, stent 640, and/or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, 314LV, 304, or 316 stainless steel; mild steel; nickel-titanium alloy such as linear-elastic and/or super-elastic nitinol; other nickel alloys such as cobalt-chromium-tungsten-nickel alloy (e.g., UNS: R30605 such as L605®), nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the catheter 30, 230, 530, 630 and the skirt 40, 240, 540, stent 640, and/or components thereof, may also be doped with, made of, or otherwise include a radiopaque material. Radiopaque materials are understood to be materials capable of producing a relatively dark image on a fluoroscopy screen or another imaging technique during a medical procedure. This relatively dark image aids a user in determining the location of the catheter 30, 230, 530, 630 and the skirt 40, 240, 540 or stent 640. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the catheter 30, 230, 530, 630 and the skirt 40, 240, 540 or stent 640 to achieve the same result.

In some embodiments, a degree of Magnetic Resonance Imaging (MRI) compatibility is imparted into the catheter 30, 230, 530, 630 and the skirt 40, 240, 540 or stent 640. For example, the catheter 30, 230, 530, 630 and the skirt 40, 240, 540, stent 640, and/or components or portions thereof, may be made of a material that does not substantially distort the image and create substantial artifacts (e.g., gaps in the image). Certain ferromagnetic materials, for example, may not be suitable because they may create artifacts in an Mill image. The catheter 30, 230, 530, 630 and the skirt 40, 240, 540, stent 640, or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the catheter 30, 230, 530, 630 and the skirt 40, 240, 540, stent 640, and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A method of imaging a vessel, comprising:
advancing a fluid directing device intravascularly to a vessel ostium, wherein the fluid directing device includes a catheter with a skirt attached adjacent a distal end of the catheter, the skirt defined by a circumferential sidewall configured to move between a collapsed state and an expanded state in which the sidewall extends radially away from the catheter;
expanding the skirt to the expanded state;
positioning a distal end of the skirt over the vessel ostium;
delivering contrast media through a lumen in the catheter, wherein the skirt prevents contrast media from passing through the sidewall; and
imaging the vessel.

2. The method of claim 1, wherein advancing the fluid directing device includes advancing the fluid directing device through at least a first vessel to the vessel ostium leading into a second vessel.

3. The method of claim 2, wherein the skirt prevents contrast media from passing through the sidewall into the first vessel.

4. The method of claim 3, wherein the catheter extends distal of the distal end of the skirt, and delivering contrast media includes delivering contrast media directly into the second vessel.

5. The method of claim 3, wherein the skirt is attached at the distal end of the catheter, wherein delivering contrast media includes delivering contrast media into the skirt.

6. The method of claim 1, wherein the distal end of the skirt include an attachment member configured to releasably engage the vessel ostium.

7. The method of claim 6, wherein the attachment member includes a plurality of hooks.

8. The method of claim 2, wherein the skirt is impermeable to blood and contrast media such that expanding the skirt to the expanded state prevents blood flow from the first vessel into the second vessel.

9. The method of claim 1, wherein the skirt includes electrically actuatable pores, the method further comprising transmitting a current to the skirt to open the electrically actuatable pores.

10. The method of claim 1, wherein the skirt includes least one sensor configured to determine proximity to tissue, wherein positioning the distal end of the skirt over the vessel ostium includes determining when the distal end of the skirt is in contact with the vessel ostium, and then delivering contrast media.

11. A method of imaging a vessel, comprising:
advancing a fluid directing device intravascularly through at least a first vessel to a vessel ostium leading into a second vessel, wherein the fluid directing device includes a catheter with a skirt attached adjacent a distal end of the catheter, the skirt defined by a sidewall configured to move between a collapsed state and an expanded state in which the sidewall extends radially away from the catheter;
expanding the skirt to the expanded state;
releasably coupling a distal end of the skirt to the vessel ostium;
delivering contrast media through a lumen in the catheter into the second vessel, wherein the skirt prevents contrast media from passing through the sidewall into the first vessel; and
imaging the second vessel.

12. The method of claim 11, wherein the catheter extends distal of the distal end of the skirt, and delivering contrast media includes delivering contrast media directly into the second vessel.

13. The method of claim 11, wherein the skirt is attached at the distal end of the catheter, wherein delivering contrast media includes delivering contrast media into the skirt.

14. The method of claim 11, wherein the distal end of the skirt include an attachment member configured to releasably engage the vessel ostium.

15. The method of claim 14, wherein the attachment member includes a plurality of hooks.

16. The method of claim 11, wherein the skirt is impermeable to blood and contrast media such that expanding the skirt to the expanded state prevents blood flow from the first vessel into the second vessel.

17. The method of claim 11, wherein the skirt includes electrically actuatable pores, the method further comprising transmitting a current to the skirt to open the electrically actuatable pores.

18. A fluid directing device, comprising:
a catheter defining a lumen and having a distal end and a proximal end; and
a skirt attached to and completely encircling a distal region of the catheter, the skirt having a proximal end attached to the catheter, and a free distal end, the skirt having a sidewall extending between the proximal and distal ends thereof, the skirt configured to move between a collapsed state and an expanded state in which the sidewall extends radially away from the catheter, the sidewall defining an interior chamber in the expanded state, wherein the sidewall includes a plurality of electrically actuatable pores allowing the sidewall to be permeable or impermeable.

19. The fluid directing device of claim 18, wherein the plurality of electrically actuatable pores are biased closed such that the skirt is configured to prevent contrast media exiting the catheter lumen from passing from the interior chamber of the skirt through the sidewall and into a vessel.

20. The fluid directing device of claim 18, wherein the proximal end of the skirt is attached proximal of the distal end of the catheter, such that the catheter extends distal of the free distal end, with the distal end of the catheter spaced apart distally from the free distal end of the skirt.
